(19) United States
(12) Patent Application Publication
(10) Pub. No.: US 2024/0424308 A1
(43) Pub. Date: Dec. 26, 2024

(54) METHOD OF IMPLANTING A STIMULATION LEAD FOR DORSAL ROOT GANGLION STIMULATION AND RELATED IMPLANT TOOL

(71) Applicant: Advanced Neuromodulation Systems, Inc., Plano, TX (US)

(72) Inventors: Geronimo Hernandez, Dallas, TX (US); Grace Liu, Plano, TX (US); Richard Ornelaz, Frisco, TX (US)

(21) Appl. No.: 18/631,737

(22) Filed: Apr. 10, 2024

Related U.S. Application Data
(60) Provisional application No. 63/522,865, filed on Jun. 23, 2023.

(51) Int. Cl. A61N 1/372; A61N 1/05; A61N 1/36

(52) U.S. Cl. CPC A61N 1/372; A61N 1/0551; A61N 1/36062; A61N 1/36071

(57) ABSTRACT

The present disclosure provides a sheath for use in delivering a lead to a dorsal root ganglion. The sheath includes an inner liner, an outer jacket, a reinforcement braid positioned between the inner liner and the outer jacket, and a marker band extending over a portion of the reinforcement braid, the marker band positioned between the reinforcement braid and the outer jacket, wherein the sheath has an outer diameter less than 0.067 inches

### CROSS-REFERENCE TO RELATED APPLICATIONS

This application claims priority to U.S. Provisional Patent Application No. 63/522,865, filed Jun. 23, 2023, which is incorporated by reference herein in its entirety.

### A. FIELD OF THE DISCLOSURE

The present disclosure relates generally to neuromodulation systems, and more particularly to sheaths for use in delivering a stimulation lead to a dorsal root ganglion.

### B. BACKGROUND

Neuromodulation is a method of treating pain symptoms by therapeutically altering activity in pain pathways with the use of an implantable device. Neuromodulation works by either actively stimulating nerves with electrical energy to produce a natural biological response or by applying targeted pharmaceutical agents in small doses directly to a site of action.

Electrical stimulation involves the application of electrodes to the brain, the spinal cord or peripheral nerves of a patient. These precisely placed electrodes are typically mounted on a lead that is connected to a pulse generator and power source, which generates the necessary electrical stimulation. A low-voltage electrical current passes from the generator to the nerve, and can either inhibit pain signals or stimulate neural impulses where they were previously absent. One of the most common types of electrical stimulation is spinal cord stimulation (SCS), which has been used as a treatment option for patients with chronic pain since the 1960s. In the last 30 years, it has become a standard treatment for patients with chronic pain in their back and/or limbs who have not found pain relief from other treatments. While the treatment does not work for everyone, many patients who qualify for neurostimulation therapy receive a reduction in overall pain. Some patients find that they can decrease their pain medication after undergoing spinal cord stimulation. Given these benefits, many individuals suffering from chronic pain find that neurostimulation positively impacts the quality of their lives.

In some instances, neuromodulation can alternatively be achieved by delivering pharmacological agents through implanted leads or catheters. In this manner, the agent can be administered in smaller doses because it does not have to be metabolized and pass through the body before reaching the target area. Smaller doses—in the range of 1/300 of an oral dose—can mean fewer side effects, increased patient comfort and improved quality of life.

However, neuromodulation is not without its risks and complications. One complication associated with the implantation of leads is lead migration which can cause loss of effective stimulation over time. During migration, the stimulation electrodes, typically at the distal end of the lead, move in relation to the nerve, creating a less desirable stimulation effect. Traditional SCS leads are positioned within the epidural space which is a largely unconfined area. In addition, such leads are typically anchored outside of the epidural space, such as to the fascia above the supraspinous ligament or to the supraspinous ligament itself. Consequently, the portion of the lead distal to the anchor is free to move along the entire length of the lead from the point of anchor to the tip in any direction within the epidural space. Such movement can reposition the lead such that stimulation is altered or even negated over time. Similarly, catheters positioned within the epidural space can also suffer from migration leading to agents being delivered outside of the target location.

Movement or migration of the lead can be caused by: 1) body motions (flexion, torsion, and so on); 2) tensile force transferred to the distal end of the lead from the proximal portion of the lead (i.e. from the anchor IPG connection point, or fascia, or ligaments); 3) gravity settling of the lead body; and/or 4) other factors. An anchor or other means to prevent migration is intended to prevent or reduce motion of the distal end of the lead due to these causes.

Improved anchoring of leads and catheters are desired. Such anchoring should be noninvasive to avoid damaging or harming the patient anatomy, particularly delicate nerve tissue and, in some instances, reversible so as to allow a revision of the system without having to access the epidural space directly to remove the lead. At least some of these objectives will be met by the present invention.

### BRIEF SUMMARY

In one aspect, a sheath for use in delivering a lead to a dorsal root ganglion of a subject is provided. The sheath includes an inner liner, an outer jacket, a reinforcement braid positioned between the inner liner and the outer jacket, and a marker band extending over a portion of the reinforcement braid, the marker band positioned between the reinforcement braid and the outer jacket, wherein the sheath has an outer diameter less than 0.067 inches.

In another aspect, a method of fabricating a sheath for use in delivering a lead to a dorsal root ganglion of a subject is provided. The method includes positioning a reinforcement braid over an inner liner, positioning a marker band over a portion of the reinforcement braid, positioning an outer jacket over the marker band and the reinforcement braid, and forming the sheath by reflowing the inner liner and the outer jacket to encapsulate the reinforcement braid and the marker band, wherein the sheath has an outer diameter less than 0.067 inches.

In yet another aspect, a housing for managing a shape of a sheath, is provided. The housing includes an upper cover, and a lower cover selectively couplable to the upper cover, wherein the upper and lower covers collectively define a retaining groove that includes a curved section and a straight section, wherein the curved section of the retaining groove is configured to maintain a shape of a curved section of the sheath while the sheath is stored within the housing, and wherein the straight section of the retaining groove is configured to pre-straighten the curved section of the sheath when the sheath is removed from the housing.

### BRIEF DESCRIPTION OF THE DRAWINGS

FIG. 1 illustrates an example stimulation system which may utilize a slack anchor.

FIGS. 2A-2D illustrate an example lead and delivery devices for accessing a target site and creating a slack anchor.

FIG. 3 illustrates an embodiment of a sheath advanced over a shaft of a lead with an internal stylet forming a first curvature.

FIG. 4 illustrates the lead with the internal stylet of FIG. 3 extending beyond the sheath forming a second curvature.

FIGS. 5A-5D illustrate an embodiment of a method of creating a slack anchor.

FIG. 6 illustrates an embodiment of a slack anchor having a serpentine shape comprising a single switchback.

FIG. 7 illustrates an embodiment of a slack anchor having a serpentine shape comprising a plurality of switchbacks.

FIG. 8 illustrates an embodiment of a slack anchor having an irregular shape.

FIG. 9 illustrates an embodiment of a slack anchor having a loop shape.

FIG. 10 illustrates an embodiment of a slack anchor comprised of a variety of serpentine and loop shapes.

FIG. 11 illustrates an embodiment of a lead having a slack anchor which has been positioned with a retrograde approach.

FIG. 12 illustrates an embodiment of a lead having slack anchor formed by an elongated tip,

FIG. 13 illustrates an embodiment of a lead comprising a shaft having areas of differing stiffness.

FIGS. 14A-14B illustrate an embodiment of a method of creating a slack anchor using the lead of FIG. 13.

FIG. 15 illustrates an example sheath.

FIG. 16 illustrates an example layer arrangement of the sheath shown in FIG. 15.

FIG. 17 illustrates a cross-section view of the distal tip of the sheath shown in FIG. 15.

FIG. 18 illustrates a reinforcement braid on an inner liner.

FIG. 19 illustrates an example structure that may be used for a marker band.

FIG. 20 illustrates an example structure that may be used for a marker band.

FIG. 21 is a perspective view of one embodiment of a sheath shape management device.

FIG. 22 is a perspective view of a lower cover of the sheath shape management device shown in FIG. 21.

FIG. 23A shows one embodiment of a sheath after manufacturing, but prior to pre-straightening.

FIG. 23B shows the sheath of FIG. 23A after pre-straightening.

### DETAILED DESCRIPTION

The present disclosure is directed to a sheath for use in delivering a lead to a dorsal root ganglion of a subject. The sheath includes an inner liner, an outer jacket, a reinforcement braid positioned between the inner liner and the outer jacket, and a marker band extending over a portion of the reinforcement braid, the marker band positioned between the reinforcement braid and the outer jacket, wherein the sheath has an outer diameter less than 0.067 inches.

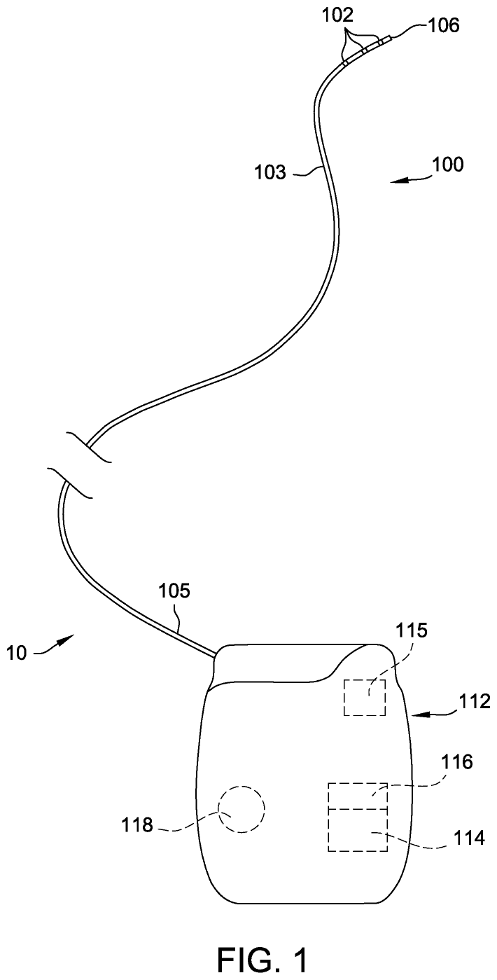

FIG. 1 illustrates an example stimulation system 10 which may utilize a slack anchor for anchoring a lead 100, such as within the epidural space of a patient. In this embodiment, the stimulation system 10 includes the lead 100, having at least one electrode 102 disposed thereon, and an implantable pulse generator (IPG) 112. The lead 100 comprises a shaft 103 having a proximal end 105 and a distal tip 106. The proximal end 105 is insertable into the IPG 112 to provide electrical connection to the lead 100. The IPG 112 contains a processor 114, an antenna 115, programmable stimulation information in memory 116, as well as a power supply 118, e.g., a battery, so that once programmed and turned on, the IPG 112 can operate independently of external hardware. The IPG 112 is turned on and off and programmed to generate the desired stimulation pulses from an external programming device using transcutaneous electromagnetic or RF links. The stimulation information includes signal parameters such as voltage, current, pulse width, repetition rate, and burst rates. Example stimulation information is provided in U.S. patent application Ser. No. 12/607,009 entitled “Selective Stimulation Systems and Signal Parameters For Medical Conditions”, filed Oct. 27, 2009, incorporated herein by reference for all purposes.

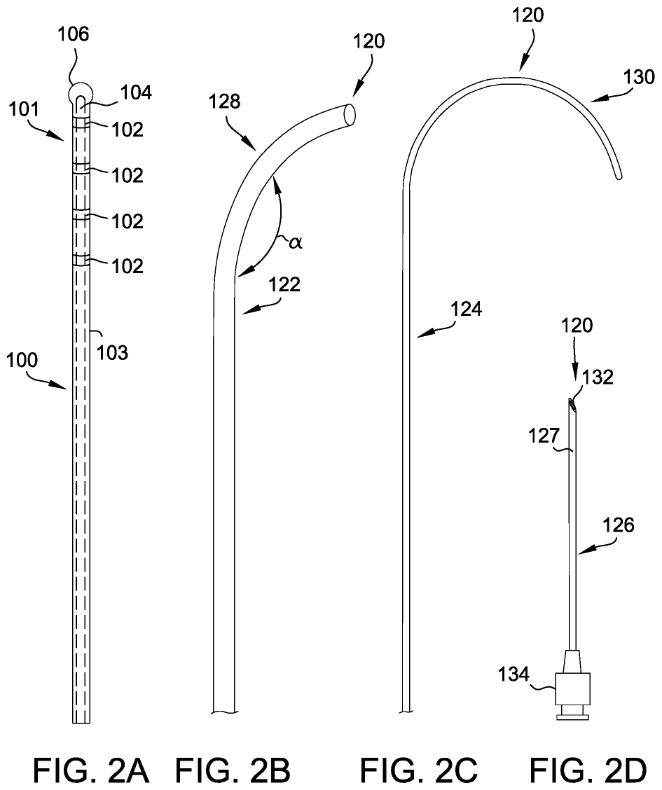

Desired positioning of the lead 100 near a target site, such as the DRG, and creation of the slack anchor may be achieved with a variety of delivery systems, devices and methods. Referring to FIGS. 2A-2D, an example lead and delivery devices for accessing a target site and creating a slack anchor are illustrated. FIG. 2A illustrates an embodiment of a lead 100 comprising a shaft 103 having a distal end 101 with four electrodes 102 disposed thereon. It may be appreciated that any number of electrodes 102 may be present, including one, two, three, four, five, six, seven, eight or more. In this embodiment, the distal end 101 has a closed-end distal tip 106. The distal tip 106 may have a variety of shapes including a rounded shape, such as a ball shape (shown) or tear drop shape, and a cone shape, and donut shape to name a few. These shapes provide an atraumatic tip for the lead 100 as well as serving other purposes. The lead 100 also includes a stylet lumen 104 which extends toward the closed-end distal tip 106. A delivery system 120 is also illustrated, including a sheath 122 (FIG. 2B), stylet 124 (FIG. 2C) and introducing needle 126 (FIG. 2D).

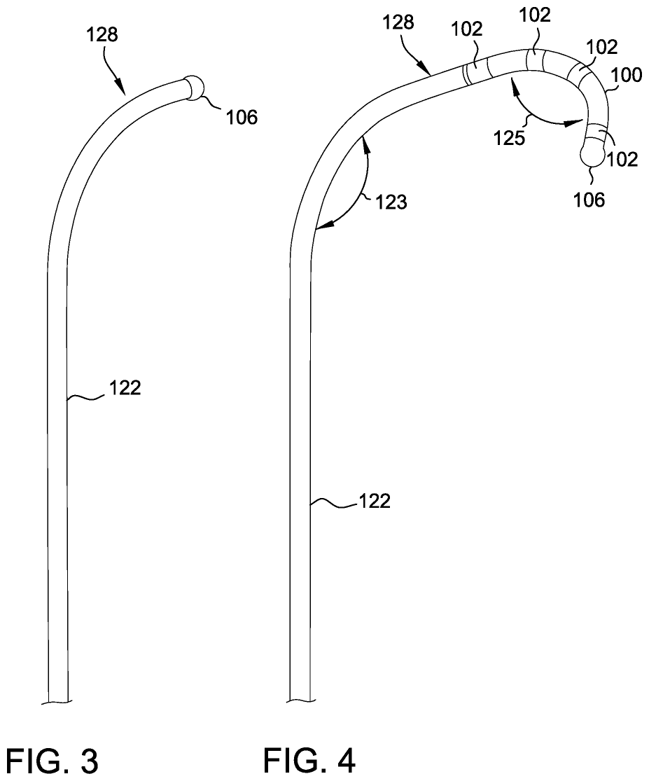

Referring to FIG. 2B, an embodiment of a sheath 122 is illustrated. In this embodiment, the sheath 122 has a distal end 128 which is pre-curved to have an angle α. In some embodiments, the angle α is in the range of approximately 80 to 165 degrees. The sheath 122 is sized and configured to be advanced over the shaft 103 of the lead 100 until a portion of its distal end 128 abuts the distal tip 106 of the lead 100, as illustrated in FIG. 3. Thus, the ball shaped tip 106 of this embodiment also prevents the sheath 122 from extending thereover. Passage of the sheath 122 over the lead 100 causes the lead 100 to bend in accordance with the precurvature of the sheath 122. Thus, when approaching a target DRG, the sheath 122 assists in steering the lead 100 along the spinal cord S and toward the target DRG, such as in a lateral direction.

Referring back to FIG. 2C, an embodiment of a stylet 124 is illustrated. The stylet 124 has a distal end 130 which is pre-curved. In some embodiments, the pre-curvature has a radius of curvature is in the range of approximately 0.1 to 0.5. The stylet 124 is sized and configured to be advanced within the stylet lumen 104 of the lead 100. Typically the stylet 124 extends therethrough so that its distal end 130 aligns with the distal end 101 of the lead 100. Passage of the stylet 124 through the lead 100 causes the lead 100 to bend in accordance with the precurvature of the stylet 124, Typically, the stylet 124 has a smaller radius of curvature, or a tighter bend, than the sheath 122. Therefore, as shown in FIG. 4, when the stylet 124 is disposed within the lead 100, extension of the lead 100 and stylet 124 through the sheath 122 bends or directs the lead 100 through a first curvature 123. Further extension of the lead 100 and stylet 124 beyond the distal end 128 of the sheath 122 allows the lead 100 to bend further along a second curvature 125. When approaching a target DRG, this allows the laterally directed lead 100 to now curve around toward the target DRG along the nerve root. This two step curvature allows the lead 100 to be successfully positioned so that at least one of the electrodes 102 is on, near or about the target DRG, particularly by making a sharp turn along the nerve root.

Thus, the lead 100 does not require stiff or torqueable construction since the lead 100 is not torqued or steered by itself. The lead 100 is positioned with the use of the sheath 122 and stylet 124 which direct the lead 100 through the two step curvature. This eliminates the need for the operator to torque the lead 100 itself and allows the lead 100 to have a lower profile as well as a very soft and flexible construction. This, in turn, minimizes erosion and discomfort created by pressure on nerve tissue, such as the target DRG and/or the nerve root, once the lead 100 is implanted. For example, such a soft and flexible lead 100 will minimize the amount of force translated to the tip of the lead 100 by body movement (e.g. flexion, extension, torsion) which in turn will reduce the variability in position of the lead with respect to the target tissue.

Referring back to FIG. 2D, an embodiment of an introducing needle 126 is illustrated. The introducing needle 126 is used to access the epidural space of the spinal cord S. The needle 126 has a hollow shaft 127 and typically has a very slightly curved distal end 132. The shaft 127 is sized to allow passage of the lead 100, sheath 122 and stylet 124 therethrough. In some embodiments, the needle 126 is 14 gauge which is consistent with the size of epidural needles used to place conventional percutaneous leads within the epidural space. However, it may be appreciated that other sized needles may also be used, particularly smaller needles such as 16-18 gauge. Likewise, it may be appreciated that needles having various tips known to practitioners or custom tips designed for specific applications may also be used. The needle 126 also typically includes a Luer-Lok™ fitting 134 or other fitting near its proximal end. The Luer-Lok™ fitting 134 is a female fitting having a tabbed hub which engages threads in a sleeve on a male fitting, such as a syringe.

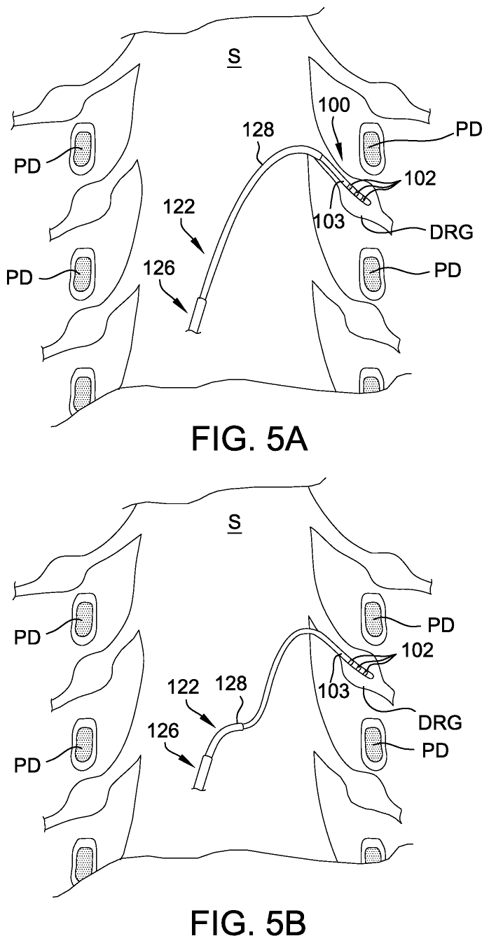

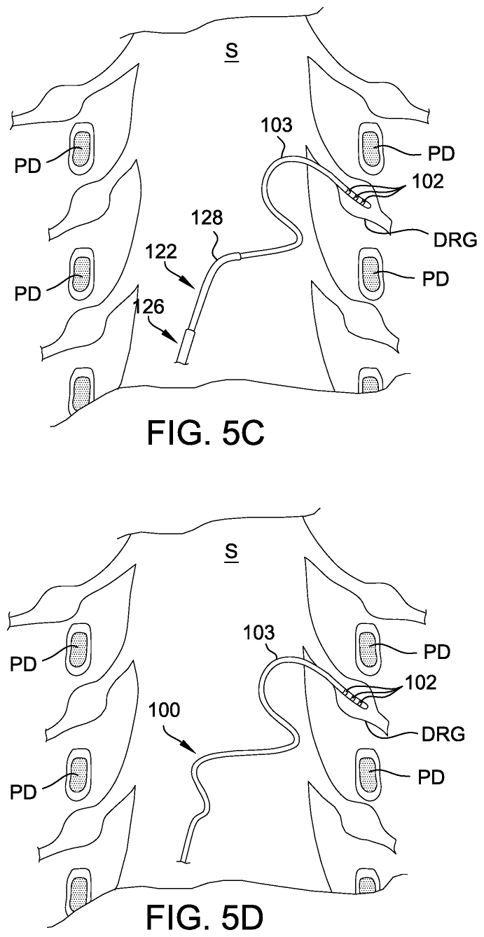

In some embodiments, the above described lead 100 and delivery system 120 is used to create a slack anchor. FIGS. 5A-5D illustrate an embodiment of a method of creating a slack anchor with the use of a lead 100 and delivery system 120 described above. In this embodiment, the lead 100 is delivered to a DRG from an antegrade approach. Each DRG is disposed along a dorsal root DR and typically resides at least partially between the pedicles PD or within a foramen. Each dorsal root DR exits the spinal cord S at an angle θ. This angle θ is considered the nerve root sleeve angulation and varies slightly by patient and by location along the spinal cord. In many instances, the nerve root angulation is significantly less than 90 degrees and sometimes less than 45 degrees. Therefore, advancement of the lead 100 toward the target DRG in this manner involves making a sharp turn along the angle θ. Turns of this severity are achieved with the use of the delivery system 120.

In this embodiment, the epidural space is accessed with the use of the introducing needle 126. Once the needle 126 has been successfully inserted into the epidural space, the lead 100 is delivered to the target DRG, as illustrated in FIG. 5A. The stylet 124 is inserted into the lead 100 and the sheath 122 is advanced over the lead 100. The sheath 122 is positioned so that its distal end 128 is near or against the distal tip 106 of the lead 100 causing the lead 100 to follow the curvature of the distal end of sheath 122. The assembled sheath 122/lead 100/stylet 124 is advanced within the epidural space toward a target DRG with the precurvature of the sheath 122 directing the lead 100 laterally outwardly. The lead 100/stylet 124 is then advanced beyond the distal end 128 of the sheath 122. The curvature of the stylet 124 within the lead 100 causes the lead 100 to bend further, along this curvature. This allows the laterally directed lead 100 to now curve around toward the target DRG along the nerve root angulation. This two step curvature allows the lead 100 to be successfully steered to position at least one of the electrodes 102 on, near or about the target DRG. Such methods of deliver are further described and illustrated in U.S. patent application Ser. No. 12/687,737, entitled “Stimulation Leads, Delivery Systems and Methods of Use”, filed Jan. 14, 2010; incorporated herein by reference for all purposes, along with examples of other delivery systems, devices and methods applicable to use with the present invention.

Thus, distal end 101 of the lead 100 is positioned at the target location and the shaft 103 extends along a first path. The sheath 122 and stylet 124 are then retracted, leaving the flexible shaft 103 of the lead 100 extending along the first path. Referring to FIG. 5B, the sheath 122 is manipulated so that the curved distal end 128 directs a portion of the shaft 103 lateral to the first path within the epidural space. FIG. 5B shows the sheath 122 directing a portion of the shaft 103 laterally outward, away from the midline of the spinal cord S. However it may be appreciated that the sheath 122 may be rotated so as to direct a portion of the shaft 103 laterally inward, toward the midline of the spinal cord S. Likewise, the sheath 122 may be manipulated so as to face a variety of other directions.

Referring to FIG. 5C, the lead 100 is then advanced beyond the curved distal end 128 of the sheath 122. Since the stylet 124 has been retracted, the shaft 103 of the lead 100 is very flexible, particularly in contrast to the sheath 122. The more rigid distal end 128 of the sheath 122 directs a portion of the flexible shaft 103 lateral to the first path so that this portion of the flexible shaft 103 resides along a second path. Thus, the difference in stiffness or flexibility between the sheath 122 and the shaft 103 of the lead 100 creates a “kink point” or bend area allowing the shaft 103 to bend and curve. This portion of curved lead 100 forms the slack anchor. Thus, the curvatures of the lead 100 provide slack and/or anchoring. The slack absorbs any movement or migration of the lead 100 within the epidural space and prevents or minimizes translation such movement to the distal end 101. This allows the distal end 101 to maintain its position and continue to provide desired stimulation to the target site. The anchoring is achieved by frictional forces created by the curvatures of the lead 100 within the epidural space and the increased surface area created by the slack. The slack and anchoring significantly reduces or eliminates the risk of migration of the leads within the epidural space.

It may be appreciated that the slack anchor may alternatively or additionally be formed with the use of the stylet 124. In such embodiments, the stylet 124 is advanced beyond the distal end 128 of the sheath 122 to a desired location within the shaft 103 of the lead 100. The stylet 124 provides increased rigidity to the shaft 103 along the areas where the stylet 124 resides within. Thus, the location where the stylet 124 ends within the shaft 103 creates a natural kink point allowing the shaft 103 to bend and curve. Consequently, the stylet 124 can be manipulated to create a variety of curvatures at any desired location along the shaft 103 of the lead 100.

In conventional spinal cord stimulation, the SCS lead is either delivered without a delivery sheath or the lead is delivered with the use of a delivery sheath which does not impart stiffness. Likewise, the lead itself is of consistent stiffness. Without a means for creating difference in stiffness, a kink point cannot be created and therefore a slack anchor cannot be easily formed.

In the present invention, a variety of different slack anchors may be formed by manipulating the sheath 122 and/or stylet 124. Once the desired slack anchor is created, the sheath 122 and stylet 124 are removed and the lead 100 is left in place, as illustrated in FIG. 5D. Since the slack anchor is disposed within the epidural space, the lead 100 is anchored as close to the target therapy site, such as the DRG, as possible. In this example, the slack anchor is formed at a location along the spinal cord, adjacent the dorsal root. By anchoring close to the target therapy site, the risk of movement or migration of the distal end 102 of the lead 100 is significantly reduced or eliminated. Such anchoring is particularly useful when accessing the epidural space on the same spinal level as the target therapy site or on a spinal level which is adjacent or nearby the target therapy site. In such instances, the distance between the entry site and the target therapy site is relatively short which increases the risk of migration. Thus, the use of a slack anchor is particularly useful in resisting migration in these instances.

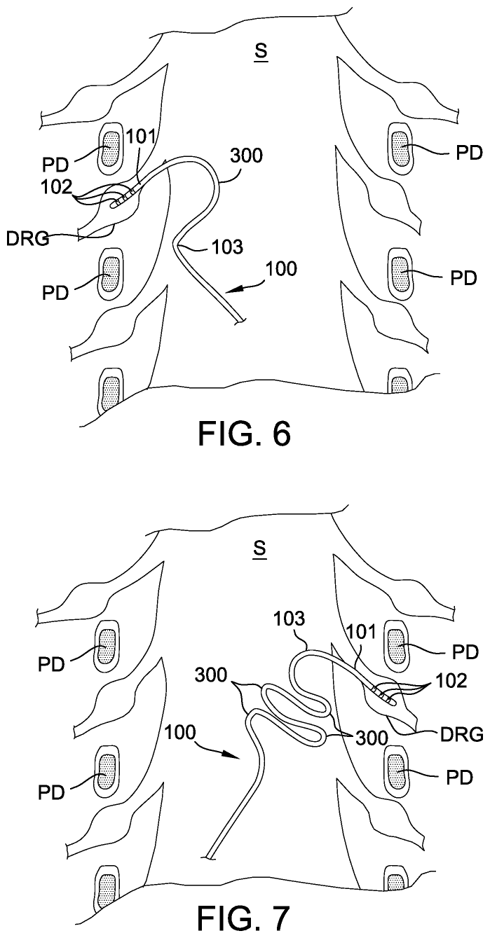

The slack anchors of the present invention may have a variety of shapes or forms. In some embodiments, the slack anchor has a serpentine shape. In such embodiments, the shaft 103 of the lead 100 curves through one or more switchbacks, such as forming an S shape, snake shape, or zigzag shape. The switchbacks may be short, such as to form wavy shapes, or long, such as to form lobe shapes. In addition, the number of switchbacks may be minimal, such as one or two, or more plentiful. FIG. 6 illustrates an embodiment of a slack anchor having a serpentine shape comprising a single switchback 300. Here, the distal end 101 of the lead 100 is positioned near a DRG and the shaft 101 extends along the nerve root angulation and along portions of the spinal cord. Had the lead 100 not included a slack anchor, the shaft 103 would reside along a first path extending toward the point of entry to the epidural space. However, in this embodiment, the shaft 103 is positioned along a second path having the serpentine shape which forms the slack anchor. FIG. 7 illustrates an embodiment of a slack anchor having a serpentine shape comprising a plurality of switchbacks 300. In this embodiment, four switchbacks 300 are present. Each switchback 300 is relatively long so as to form lobe shapes.

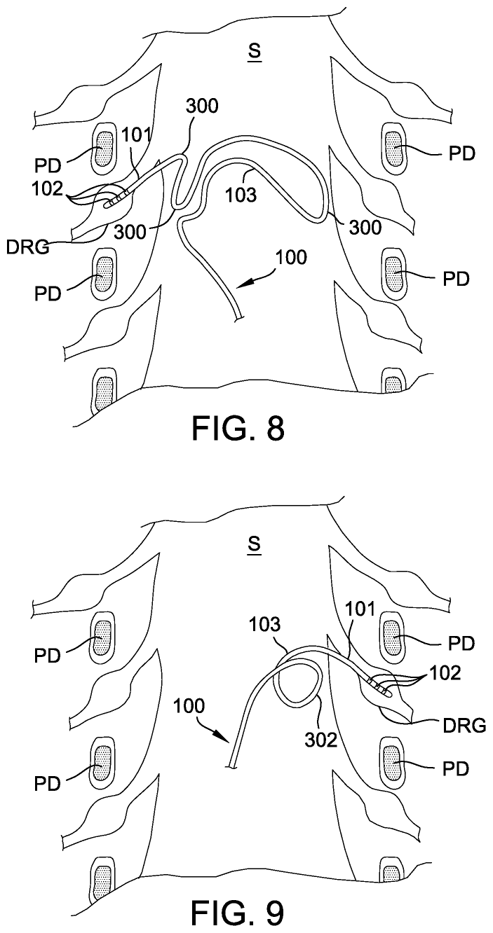

In some embodiments, the slack anchor has an irregular shape, such as a combination of shapes. For example, FIG. 8 illustrates an embodiment of a slack anchor having an irregular shape. Here, the distal end 101 of the lead 100 is positioned near a DRG and the shaft 101 extends along the nerve root angulation into the spinal area of the spinal cord S. Again, had the lead 100 not included a slack anchor, the shaft 103 would reside along a first path extending toward the point of entry to the epidural space. However, in this embodiment, the shaft 103 is positioned along a second path having the irregular shape which forms the slack anchor. The second path includes a serpentine shape, wherein the shaft 103 extends through two small switchbacks 300. The second path then extends across the epidural space forming a large switchback or lobe 300′ before extending toward the point of entry. In this embodiment, the slack anchor extends across the width of the spinal cord S providing significant slack and anchoring capabilities.

In some embodiments, the slack anchor has a loop shape. For example, FIG. 9 illustrates an embodiment of a slack anchor having a loop shape. Here, the loop shape is formed by creating a switchback that crosses over itself forming a loop 302. As shown in FIG. 9, the distal end 101 of the lead 100 is positioned near a DRG and the shaft 101 extends along the nerve root angulation into the spinal column. The shaft 101 begins along a first path and then extends along a second path having a loop shape. In this embodiment, the loop 302 extends away from the midline of the spinal cord S. However, it may be appreciated that in some embodiments the loop 302 extends toward the midline of the spinal column S. Likewise, it may be appreciated that any number of loops 302 may be present and the loops 302 may be of any size.

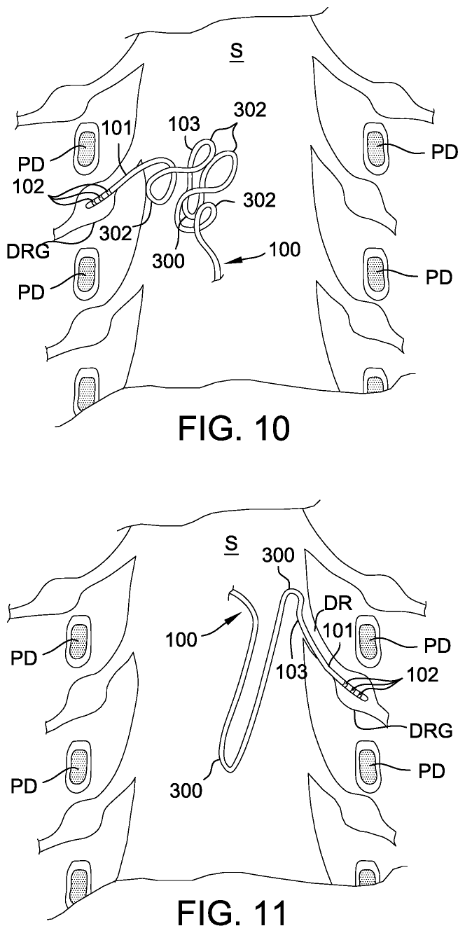

In some embodiments, the slack anchor has a combination of serpentine and loop shapes. For example, FIG. 10 illustrates an embodiment of a slack anchor comprised of a variety of serpentine and loop shapes. In this embodiment, the slack anchor includes as least four loops 302, wherein some of the loops 302 cross over underlying switchbacks 300. Thus, the shaft 103 of the lead 100 follows a complex path forming the slack anchor.

In some embodiments, the slack anchor is configured to allow atraumatic removal of the lead 100 from the epidural space after the slack anchor has been formed. The epidural space is comprised of fluid and fibrous connective tissue. Fibrous tissue forms around the lead 100 over time creating a biological structure within the epidural space. The path of the lead 100 is essentially a tunnel or passageway through the biological structure so the lead 100 is able to move freely, and therefore migrate. However, the slack anchors of the present invention are supported by the biological structure so that the tunnels or passageways follow the curves and contours of the slack anchor path. Since the slack anchor path is non-linear, such as serpentine, the lead 100 is held in place by the biological structure and migration is reduced. In addition, if it is desired to remove the lead 100, the lead 100 may be withdrawn from the epidural space by gently pulling the proximal end of the lead 100 until the lead 100 is removed. The lead 100 will move through the tunnels or passageways, following the curves and contours of the slack anchor path. Such movement may be achieved with the force of withdrawal, however such movement is not achieved with the mere forces of migration. It may be appreciated that in some embodiments the slack anchor is configured to remain as a permanent anchor wherein the lead 100 is not easily removable after the biological structure has formed therearound. Such slack anchors are typically convoluted or complex resisting easy withdrawal of the lead 100 through the path.

It may be appreciated that although the epidural delivery methods described above illustrate an antegrade approach to a target site accessible through the epidural space, a variety of other approaches may also be used. For example, a retrograde, contralateral or transforaminal approach may be used, to name a few. FIG. 11 illustrates an embodiment of a lead 100 which has been positioned with a retrograde approach. Here the target site is the DRG and the lead 100 is positioned so that the at least one electrode is in the vicinity of the DRG. Thus, the distal end 101 of the lead 100 extends along the dorsal root DR and into the area of the spinal cord S where a slack anchor is formed by the shaft 103 of the lead 100. In this embodiment, the slack anchor is comprised of two switchbacks 300. Leads 100 positioned with this approach benefit greatly from the presence of a slack anchor since the first path of the lead 100 is often substantially linear which can have very little resistance to migration.

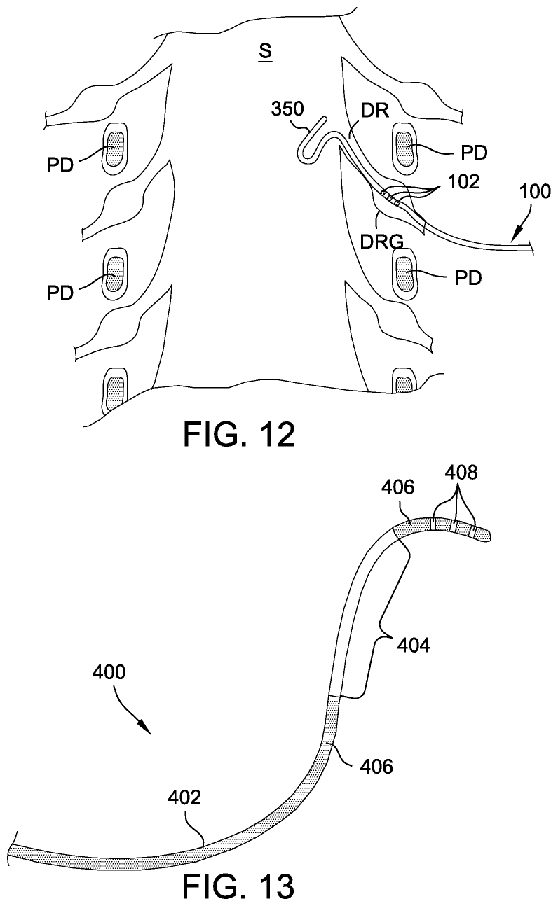

FIG. 12 illustrates an embodiment of a lead 100 which has been positioned with a transforaminal/extraforaminal approach, wherein the DRG is approached from outside of the spinal column. In this embodiment, the lead 100 has an elongated distal tip 350 so that the distal tip 350 extends into the area of the spinal cord S while the at least one electrode 102 resides in proximity to the DRG. Here, the slack anchor is formed by the elongated distal tip 350 so as to anchor the lead 100 within the epidural space. Such a slack anchor may be formed with any of the techniques described above, such as with the use of the sheath 122 and/or stylet 124.

It may also be appreciated that the slack anchors of the present invention may be formed by leads and devices provided in U.S. Provisional Patent Application No. 61/178,847, entitled “Methods, Systems and Devices for Delivering Stimulation to Spinal Anatomy, filed on May 15, 2009, incorporated herein by reference for all purposes. Likewise, the slack anchors of the present invention may be used to anchor such leads and devices positioned with the methods described therein.

In some embodiments, a modified lead 400 is used to create a slack anchor. In these embodiments, the lead 400 includes a structural kink point or bend area which assists in the creation of the slack anchor. For example, in some embodiments the structural kink point comprises a geometric feature, such as uv-notch. In other embodiments, the kink point comprises a change in material stiffness. For example, in some embodiments, the lead 400 comprises a shaft 402 having areas of differing stiffness, such as illustrated in FIG. 13. Here, the shaft 402 includes a flexible region 404 disposed between more rigid regions 406 (indicated by shading). Since the flexible region 404 is the area within which the slack anchor will be formed, the flexible region 404 is typically located proximal and close to the at least one electrode 408. Thus, the at least one electrode 408 will be anchored close to the target stimulation site.

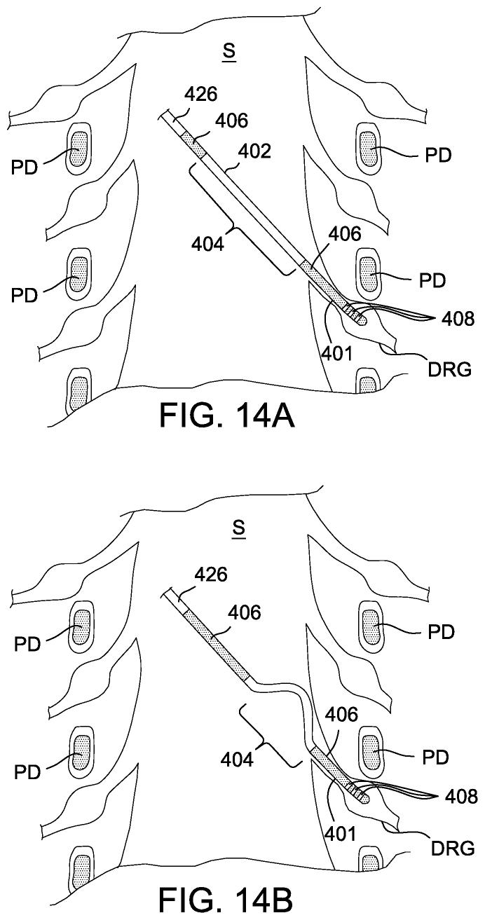

FIG. 14A illustrates the lead of FIG. 13 positioned near a target treatment site, in this instance a DRG. In this embodiment, the lead 400 is delivered to the DRG from a contralateral approach. The epidural space is accessed with the use of an introducing needle 426 and the lead 400 is advanced toward the target DRG so that the at least one electrode 408 is desirably positioned in relation to the target DRG. Thus, the distal end 401 of the lead 400 is positioned at the target location and the shaft 402 extends along a first path. Referring to FIG. 14B, the shaft 402 is then advanced through the introducing needle 426 along the first path due to the rigidity of the proximal more rigid region 406. However, this force is not significantly translated to the distal end 401 of the lead 400 due to the flexible region 404 therebetween, and the flexible region 404 bends or curves along a second path which typically includes portions which are lateral to the first path. Thus, the flexible region 404 forms a slack anchor and resists translation of motion to the distal end 401 of the lead 400. This assists in anchoring and prevention of lead migration.

It may be appreciated that forming a slack anchor in this manner, without the use of a sheath and/or stylet, is typically a less controlled method. The bends and curves formed in the flexible region are typically a product of the lead configuration in combination with the anatomical environment, wherein the user has less control over the actual shape of the slack anchor. In contrast, formation of a slack anchor with the use of a sheath and/or stylet, as described above, allows the user detailed control over each contour of the slack anchor.

A change in material stiffness along a lead 400, such as described and illustrated in relation to FIG. 13, can be created by a variety of methods or techniques. In some embodiments, the lead 400 has a construction as described and illustrated in U.S. patent application Ser. No. 12/687,737, entitled “Stimulation Leads, Delivery Systems and Methods of Use”, filed Jan. 14, 2010, incorporated herein by reference for all purposes. In particular, in some embodiments the shaft 402 of the lead 100 is comprised of single lumen tube formed from an extruded polymer, such as urethane. Additional elements, such as conductor cables and optionally a tensile element, extend through the single lumen tube. In such embodiments, the shaft 402 is potted with a harder material to create the more rigid regions 406 of the lead 400. When the shaft 402 is comprised of a soft durometer material, such as polyurethanes (e.g. Bionate, Pellethane) or silicone, the potting material is comprised of a material having a relatively higher stiffness, such as epoxy (e.g. Epotek). The potting material is injected or deposited within the single lumen tube, surrounding the elements extending therethrough, and allowed to harden. This potting material increases the stiffness of the lead 400 in the areas within which it is deposited. Therefore, specific more rigid regions 406 may be created anywhere along the lead 400. In some embodiments, the lead 400 is potted in all areas except for the area within which the slack anchor is formed. In other embodiments, the lead 40 is potted proximally, leaving the distal-most end of the lead unspotted and more flexible. For example, in some embodiments where the lead 400 has a length of approximately 40 cm, the most proximal 30 cm of the lead 400 are potted.

It may be appreciated that particular portions of the lead 400, such as the distal end 401, may be preformed into a curve so as to more easily access a DRG (particularly through an antegrade approach). Pre-curving of potted areas may be achieved by pre-curving the shaft 402 prior to hardening of the potting material therein so that the hardened potting material sets the precurvature. Such precurvature may be useful when delivering the lead 400 without the use of a sheath or stylet. In addition, in such embodiments the lead 400 may not include a stylet lumen which reduces the outer diameter, such as up to approximately 25-40%. Such reduction in diameter may increase the ability to access particular anatomy, such as stenosed foraminal openings or peripheral nerves

In other embodiments, the shaft 402 is interoperatively tilled with a deployable curing polymer to create the more rigid regions 406 of the lead 400. Again, in some embodiments the shaft 402 of the lead 100 is comprised of single lumen tube formed from an extruded polymer, such as urethane. Additional elements, such as conductor cables and optionally a tensile element, extend through the single lumen tube. In such embodiments, the shaft 402 is injected with a polymer or other material that cures to create the more rigid regions 406 of the lead 400. This cured material increases the stiffness of the lead 400 in the areas within which it is deposited. Since the material is injected interoperatively, the user is able to determine the desired locations for the more rigid regions 406 based on the specific anatomy of the patient and on the particulars of the surgical procedure. Thus, the location and configuration of the slack anchor may be precisely individualized for the patient.

It may be appreciated that a change in material stiffness along a lead 400 can alternatively be created by a variety of other methods or techniques. For example, the wall of the shaft 402 may be reinforced in the more rigid regions 406, such as by a harder durometer material, a reinforcing braid or straight wire composite, co-extrusion with a second stiffer material, overmolding, or thickening of the wall, to name a few. Likewise, the shaft 402 may be comprised of a variety of materials, each having a different durometer. For example, the shaft 402 may be comprised of single lumen tube having a stiffer durometer in the more rigid regions 406 and a less stiff durometer in the flexible regions 404. There are several scales of durometer, each used for materials with different properties. The two most common scales, using slightly different measurement systems, are the ASTM D2240 type A and type D scales. The A scale is for softer plastics, while the D scale is for harder ones. However, the ASTM D2240-00 testing standard calls for a total of 12 scales, depending on the intended use: types A, B, C, D, DO, E, M, O, OO, OOO, OOO-S, and R. Each scale results in a value between 0 and 100, with higher values indicating a harder material. Thus, the use of materials having widely differing values, such as a “C” durometer 55 and 70, may be used to create a kink point according to the present invention.

In other embodiments, a change in material stiffness along the lead 400 is created by a separable stylet. In such embodiments, the stylet is first used to assist in positioning the lead 400, such as described above. Once the lead 400 has been desirably positioned, the stylet is separated, divided, disjoined or decoupled so as to leave a portion of the stylet within the lead 400 forming a more rigid region 406. The area having the stylet removed therefrom forms the flexible region 404. For example, in some embodiments the stylet extends to or near the distal tip of the lead 400 wherein the stylet is separable at a location proximal to the distal tip. The stylet is then pulled back a desired distance to create a flexible region wherein which a slack anchor is formable. The remainder of the stylet then resides proximal to this flexible region so as to create a lead having a change in material stiffness such as illustrated in FIG. 13. A slack anchor may then be created, such as according to methods similar to the methods illustrated in FIGS. 14A-14B, It may be appreciated that the stylet may be separable in a variety of locations so as to create various patterns of more rigid regions 406. It may also be appreciated that the stylet may be used for the purpose of creating material stiffness, without the use of positioning the lead.

Similarly, in some embodiments a change in material stiffness along the lead 400 is created by a separable sheath. In such embodiments, the sheath is first used to assist in positioning the lead 400, such as described above. Once the lead 400 has been desirably positioned, the sheath is separated, divided, disjoined or decoupled so as to leave a portion of the sheath along the lead 400 forming a more rigid region 406. The area having the sheath removed therefrom forms the flexible region 404. For example, in some embodiments the sheath extends near the distal tip of the lead 400 proximal to the electrodes, wherein the sheath is separable at a location proximal to the distal end of the sheath. The sheath is then pulled back a desired distance to create a flexible region wherein which a slack anchor is formable. The remainder of the sheath then resides proximal to this flexible region. A slack anchor may then be created, such as according to methods similar to the methods illustrated in FIGS. 14A-14B. It may be appreciated that the sheath may be separable in a variety of locations so as to create various patterns of more rigid regions 406. It may also be appreciated that the sheath may be used for the purpose of creating material stiffness, without the use of positioning the lead.

It may be appreciated that the devices, systems and methods described herein may be used to reduce lead migration in leads targeting any portion of the nervous system. Leads may be positioned so as to stimulate portions of the central nervous system, such as the spinal cord, spinal nerves, and brain. Likewise, leads may be positioned so as to stimulate portions of the peripheral nervous system. In particular, leads may be positioned as described in U.S. Provisional Patent Application No. 61/473,132 entitled “Devices, Systems and Methods for Modulation of the Nervous System,” filed Apr. 7, 2011, incorporated herein by reference for all purposes. To reduce the potential for lead migration in any of these lead positions, a slack anchor may be formed along the lead according to any of the methods described herein. Such a slack anchor may be positioned within the epidural space. Or, the slack anchor may be formed outside of the epidural space. In some embodiments, when creating a slack anchor in tissue outside of the epidural space, a virtual space is created in the tissue with the use of a variety of space generating techniques, such as with the use of expanders, retractors, dissectors, tunneling tools, and insufflators to name a few. The slack anchor is then created within the virtual space providing strain relief and anchoring capabilities which assist in maintaining the position of the distal end of the lead near the target tissue. In other embodiments, when creating a slack anchor in tissue outside of the epidural space, naturally existing spaces are utilized for positioning a slack anchor therein.

As described above, delivering a lead to a target DRG may be accomplished using a sheath with a curved geometry, such as the sheath 122 shown in FIG. 2B. To facilitate lead delivery, it is desirable to reduce the size of the sheath while still maintaining appropriate stiffness of the sheath. Suitable stiffness of the sheath may be achieved by reinforcing the sheath with a metal braid layer. Free braid ends of the metal braid layer may be collared with a radiopaque marker band to prevent exposure of those free braid ends during manufacturing of the sheath. However, a rigid radiopaque marker band may stress an encapsulating material of the sheath, particularly when the rigid radiopaque marker band is located in a curved section of the sheath. As the length of the marker band increases and the radius of curvature of the curved section decreases, the resulting stress increases, which may reduce the mechanical robustness of the sheath. Accordingly, a sheath including a marker band that reduces imparted stresses would be desirable.

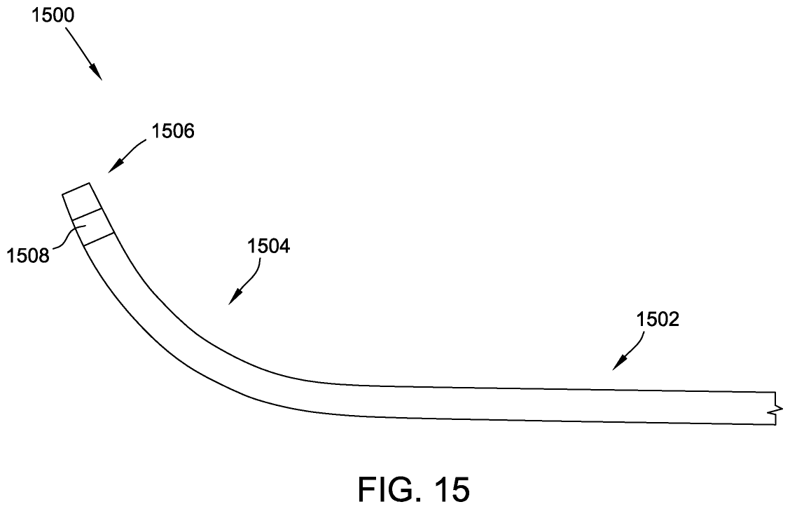

FIG. 15 illustrates an example sheath 1500. The sheath 1500 includes a curved section 1502 and a straight section 1504 proximal of the curved section 1502. A distal tip 1506 is located at an end of the curved section 1502. The curved section 1502 also includes a marker band 1508. The material selection and design of the sheath 1500 (as detailed herein) improves the ability of the sheath 1500 to maintain a curved shape even after insertion through a delivery needle. The sheath 1500 also provides sufficient flexural stiffness, kink resistance, and torsional stiffness to facilitate delivering a lead to a target DRG.

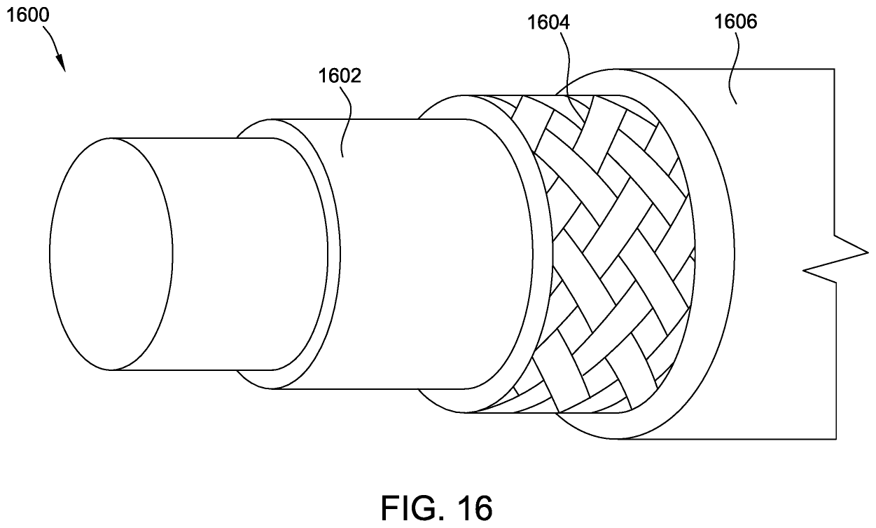

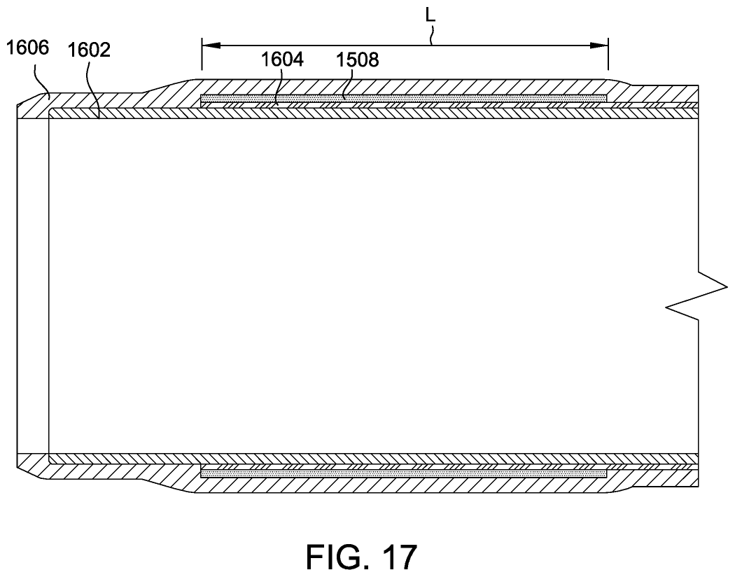

FIG. 16 illustrates an example layer arrangement 1600 of the sheath 1500 shown in FIG. 15, and FIG. 17 illustrates a cross-sectional view of the distal tip 1506 of the sheath 1500 shown in FIG. 15, including the marker band 1508. As shown in FIGS. 16 and 17, the layer arrangement 1600 includes an inner liner 1602, a reinforcement braid 1604, and an outer jacket 1606. The reinforcement braid 1604 is positioned between the inner liner 1602 and the outer jacket 1606.

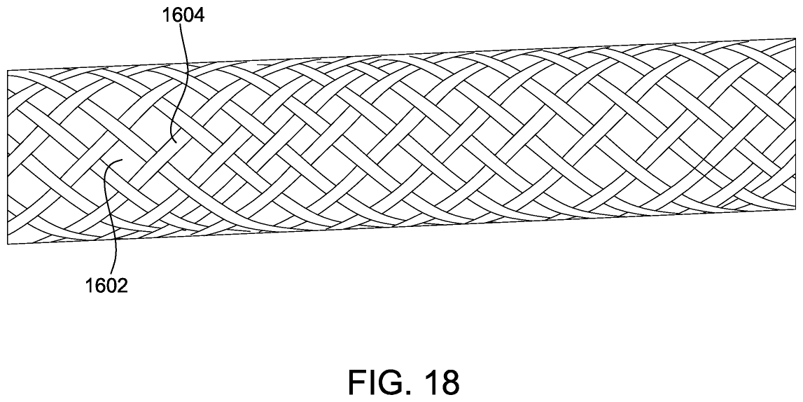

As shown in FIG. 17, the marker band 1508 has a length, L, and extends over a portion of the reinforcement braid 1604. The marker band 1508 may be made of a radiopaque material. Further, the marker band 1508 is positioned between the reinforcement braid 1604 and the outer jacket 1606. FIG. 18 illustrates the reinforcement braid 1604 on the inner liner 1602 (with the outer jacket 1606 omitted).

In at least some embodiments, the inner liner 1602 and the outer jacket 1606 are each made of a suitable polymer material. To form the sheath 1500, the polymer materials of the inner liner 1602 and the outer jacket 1606 are reflowed to encapsulate the reinforcement braid 1604 and the marker band 1508. In one embodiment, the inner liner 1602 and/or the outer jacket 1606 are made from polyamide 12, which provides appropriate stiffness. For example, the inner liner 1602 and at least a portion of the outer jacket 1606 may be polyamide 12 doped with 20-30% weight/weight BaSO₄. The BaSO₄ doping stiffens the sheath 1500 without having to increase the dimensions of the sheath.

In some embodiments, the material of the outer jacket 1606 in the straight section 1504 is a different, stiffer material than the material of the outer jacket 1606 in the curved section 1502. For example, in the straight section 1504, the outer jacket 1606 may be made of polyamide 12 doped with 20-30% weight/weight BaSO₄. In contrast, in the curved section 1502, the outer jacket 1606 may be made from block copolymers with a nominal durometer of 70 D or greater, including rigid polyamide blocks and soft polyether blocks doped with 20-30% weight/weight BaSO₄ (alternatively, the block copolymers may be undoped). This facilitates significant curve rebound after the sheath 1500 is inserted through an access needle. Further, the material of the inner liner 1602 facilitates reducing friction when deploying the lead.

In one embodiment, the sheath 1500 has a relatively thin wall (e.g., with a thickness less than 0.008 inches (0.2032 millimeters (mm))). The inner liner 1602 may have a thickness that is less than 40% of the thickness of the outer jacket 1606. Further, in one embodiment, the sheath 1500 has an outer diameter less than 0.067 inches (1.702 mm). The relatively small outer diameter provides flexural stiffness, kink resistance, and torsional stiffness to facilitate delivering a lead to a target DRG. The small diameter also facilitates reducing trauma to the patient.

The reinforcement braid 1604 may be made of steel. In one example, the reinforcement braid 1604 includes sixteen steel wires (e.g., 0.001×0.005 steel wires) braided in a 1 under 1 over 1 pattern, with 38-52 picks per inch (PPI). Alternatively, any suitable wires may be used for the reinforcement braid 1604. For example, in some embodiment, a 1 under 2 over 2 braid pattern is used.

As described herein, in some embodiments, the marker band 1508 is made of a flexible metal. For example, the marker band 1508 may be made of a metal having a density greater than 6.45 grams per cubic centimeter (g/cm³).

The flexibility of the marker band 1508 allows the marker band 1508 to conform to the curvature of the curved section 1502, which reduces stress on the outer jacket 1606 without comprising the ability of the marker band 1508 to secure the wire ends of the reinforcement braid 1604. The flexibility of the marker band 1508 also enables using a longer marker band with negligible increases in induced stress. For example, in some embodiments, the marker band 1508 has a length between 0.020 inches (0.508 mm) and 0.040 inches (1.016 mm) to facilitate capturing wire ends of the reinforcement braid 1604 and forming a curved shape without damaging the outer jacket 1606. Of note, the marker band 1508 is still short enough to form curves as tight as a curve having a radius of curvature of 0.2 inches (5.08 mm).

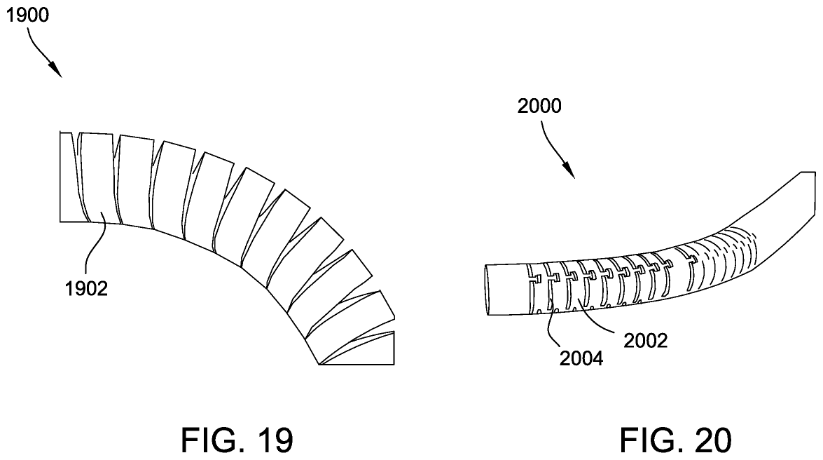

Further, the marker band 1508 may have a flexible architecture to promote flexibility. For example, in one embodiment, similar to a structure 1900 shown in FIG. 19, the marker band 1508 is formed from a spiral winding of a flat wire 1902. Free ends of the flat wire 1902 may be joined to prevent unraveling. The flat wire may have a thickness, for example, of less than 0.00125 inches (0.0318 mm). In another embodiment, similar to a structure 1900 shown in FIG. 20, the marker band 1508 is formed from a cylinder 2002 with patterns 2004 (e.g., slits) defined in the cylinder 2002 to promote flexibility. The cylinder 2002 may have a thickness, for example, of approximately 0.00125 inches (0.0318 mm).

Using the embodiments described herein enables having a smaller radius of curvature in the curved section of the sheath. Notably, the smaller radius of curvature may result in new challenges in the design of the sheath. For example, it is generally desirable to ensure that the geometry of the sheath (including the smaller radius of curvature) remains fixed during the shelf life of the device, and does not change (e.g., due to exposure to high temperatures and/or polymer aging). Further, to reduce the effort required to insert the sheath into the straight access needle, it would be desirable to pre-straighten the curved section of the sheath prior to insertion.

Accordingly, this disclosure also provides a multi-purpose sheath shape management device that facilitates both i) retaining the shape of the curved section during the shelf life and ii) pre-straightening the curved section for simplified insertion into the access needle.

The sheath shape management device is a protective housing that manages the shape of the curved section of the sheath from production until use (i.e., during the shelf life), and that also facilitates pre-straightening the curved section for simplified insertion into the access needle. As described herein, the sheath shape management device includes an internal support structure that secures the sheath in a desired geometry. Further, the sheath shape management device enables loading and locking the sheath into the housing without having to straighten or deform the curved section. In addition, the sheath shape management device pre-straightens the curved section prior to use without damaging the sheath by forcing the curved section through a straight section of the housing.

The sheath shape management device advantageously protects the shape of the curved section post manufacturing, through the shelf life, until clinical use. Further, it provides a simple means of pre-straightening the curved shape prior to insertion into the introducing needle. In addition, in at least some embodiments, the housing is opaque, which obscures the initial (i.e., prior to the pre-straightening) shape of the curved section. This may be beneficial, as the initial shape curved section may have a tighter radius of curvature than at least some known sheaths that clinicians are familiar with.

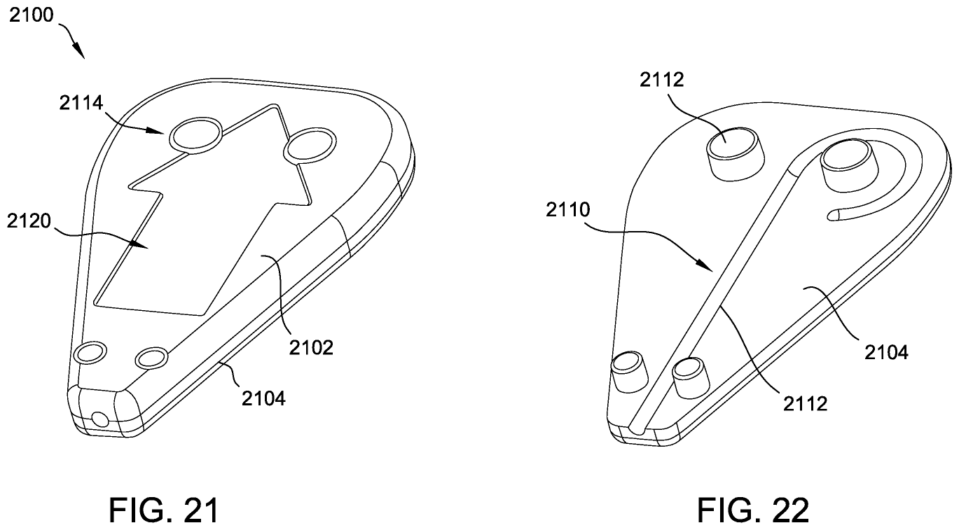

FIG. 21 is a perspective view of one embodiment of a sheath shape management device 2100. The sheath shape management device 2100 includes an upper cover 2102 and a lower cover 2104. FIG. 22 is a perspective view of the lower cover 2104. The upper and lower covers 2102 and 2104 may be made of, for example, a suitable plastic.

The sheath itself may be formed of a thermoplastic polymer belonging to the polyamide family (e.g., nylon, Pebax, and derivatives thereof). However, thermoplastic polymers are generally deformable and even meltable under sufficient heat. To prevent deformation of the sheath, should it encounter increased temperatures, the sheath shape management device 2100 restrains the curved section of the sheath during the shelf life. Specifically, as shown in FIG. 22, the lower cover 2104 defines a retaining groove 2110.

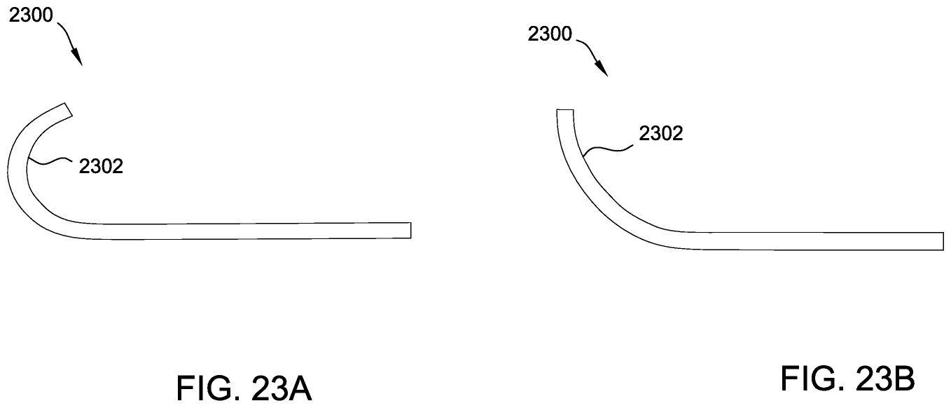

FIG. 23A shows one embodiment of a sheath 2300 after manufacturing, but prior to pre-straightening. FIG. 23B shows the sheath 2300 after pre-straightening. As shown in FIGS. 23A and 23B, the sheath 2300 initially has a relatively tight radius of curvature in a curved section 2302 (forming a shepherd's hook-like shape). After pre-straightening, however, the radius of curvature in the curved section 2302 is larger.

Returning back to FIG. 22, the retaining groove 2110 matches the initial shape of the sheath 2300. After manufacturing, the sheath 2300 is placed into the retaining groove 2110 on lower cover 2104. Next, the sheath shape management device 2100 is closed by placing the upper cover 2102 onto the lower cover 2104.

In this embodiment, the lower cover 2104 includes one or more locking pins 2112 that engage corresponding apertures 2114 formed in the upper cover 2102. This facilitates aligning the upper and lower covers 2102 and 2104 when closing the sheath shape management device 2100. Further, the locking pins 2112 may engage the corresponding apertures 2114 with a press fit engagement, locking the upper and lower covers 2102 and 2104 together.

When the clinician is ready to use the sheath 2300, rather than open the sheath shape management device 2100, the clinician slides the closed sheath shape management device 2100 off of the sheath 2300. To assist the clinician, in at least some embodiments, the upper cover 2102 defines an arrow-shaped recess 2120 that indicates which way the sheath shape management device 2100 should be slid. Alternatively, any suitable marking or indicia may be used to indicate the direction for sliding the sheath shape management device 2100. For example, in one embodiment, the entire sheath shape management device 2100 is arrow-shaped.

Sliding the sheath shape management device 2100 off of the sheath 2300 forces the curved section of the sheath 2300 through a straight section 2122 of the retaining groove 2110. This pre-straightens the curved section of the sheath 2300.

Accordingly, the retaining groove 2110 maintains and secures the shape of the sheath 2300 during the shelf life. The two-piece design of the sheath shape management device 2100 enables loading the sheath into the retaining groove 2110 without having to deform the sheath 2300. Further, the locking pins 2112 aid in closing and locking the sheath shape management device 2100. Then, removing the sheath 2300 from the sheath shape management device 2100 forces the curved section of the sheath 2300 through the straight section 2122 of the retaining groove 2110, pre-straightening the sheath 2300 without damaging the sheath 2300.

As an alternative to using the sheath shape management device 2100, the pre-straightening of the sheath 2300 may be done as a final manufacturing step. However, in this scenario, the sheath 2300 may deform during the shelf life if exposed to elevated temperatures. Accordingly, using the sheath shape management device 2100 robustly maintains the shape of the sheath 2300 during the shelf life.

In some embodiments, additional features may be added to the sheath shape management device 2100. For example, the sheath shape management device 2100 may include features that enable supplying sterilization gases to the sheath 2300, features that aid in manufacturing or packaging the sheath shape management device 2100, and/or marking to indicate proper use or cautionary information.

The sheath shape management device 2100 may be made of thermoplastic materials that are stable during ethylene oxide sterilization, such as ABS, fluorine-based polymers, polyesters, polyamides, polyacetals, polypropylene, polyethylene, PVC, polystyrene, and polymethylmethacrylate.

The embodiments described herein provide systems and methods for a sheath for use in delivering a lead to a dorsal root ganglion of a subject. The sheath includes an inner liner, an outer jacket, a reinforcement braid positioned between the inner liner and the outer jacket, and a marker band extending over a portion of the reinforcement braid, the marker band positioned between the reinforcement braid and the outer jacket, wherein the sheath has an outer diameter less than 0.067 inches.

Although the foregoing invention has been described in some detail by way of illustration and example, for purposes of clarity of understanding, it will be obvious that various alternatives, modifications, and equivalents may be used and the above description should not be taken as limiting, in scope of the invention which is defined by the appended claims.

All publications and patent applications mentioned in this specification are herein incorporated by reference to the same extent as if each individual publication or patent application was specifically and individually indicated to be incorporated by reference.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A sheath for use in delivering a lead to a dorsal root ganglion of a subject, the sheath comprising:
an inner liner;
an outer jacket;
a reinforcement braid positioned between the inner liner and the outer jacket; and
a marker band extending over a portion of the reinforcement braid, the marker band positioned between the reinforcement braid and the outer jacket, wherein the sheath has an outer diameter less than 0.067 inches.

2. The sheath of claim 1, wherein the sheath comprises a straight section and a curved section distal of the straight section, and wherein the marker band is included within the curved section.

3. The sheath of claim 2, wherein the outer jacket extends through the straight section and the curved section, and wherein the outer jacket is made of a first material in the curved section and made of a second, stiffer material in the straight section.

4. The sheath of claim 1, wherein the sheath has a wall thickness of less than 0.008 inches.

5. The sheath of claim 1, wherein the inner liner has a first thickness, wherein the outer jacket has a second thickness, and wherein the first thickness is less than 40% of the second thickness.

6. The sheath of claim 1, wherein the reinforcement braid comprises sixteen steel wires braided at 38-52 picks per inch.

7. The sheath of claim 1, wherein the reinforcement braid includes a 1 under 1 over 1 pattern.

8. The sheath of claim 1, wherein at least one of the inner liner and the outer liner are made of polymer doped with BaSO₄.

9. The sheath of claim 1, wherein the marker band has a density greater than 6.45 grams per cubic centimeter.

10. A method of fabricating a sheath for use in delivering a lead to a dorsal root ganglion of a subject, the sheath comprising:
positioning a reinforcement braid over an inner liner;
positioning a marker band over a portion of the reinforcement braid;
positioning an outer jacket over the marker band and the reinforcement braid; and
forming the sheath by reflowing the inner liner and the outer jacket to encapsulate the reinforcement braid and the marker band, wherein the sheath has an outer diameter less than 0.067 inches.

11. The method of claim 10, wherein the sheath includes a straight section and a curved section distal of the straight section, and wherein the marker band is included within the curved section.

12. The method of claim 10, wherein at least one of the inner liner and the outer liner are made of polymer doped with BaSO₄.

13. The method of claim 10, wherein the sheath has a wall thickness of less than 0.008 inches.

14. The method of claim 10, wherein the marker band has a density greater than 6.45 grams per cubic centimeter.

15. The method of claim 10, wherein the inner liner has a first thickness, wherein the outer jacket has a second thickness, and wherein the first thickness is less than 40% of the second thickness.

16. The method of claim 10, wherein the reinforcement braid comprises sixteen steel wires braided at 38-52 picks per inch.

17. A housing for managing a shape of a sheath, the housing comprising:
an upper cover; and
a lower cover selectively couplable to the upper cover, wherein the upper and lower covers collectively define a retaining groove that includes a curved section and a straight section, wherein the curved section of the retaining groove is configured to maintain a shape of a curved section of the sheath while the sheath is stored within the housing, and wherein the straight section of the retaining groove is configured to pre-straighten the curved section of the sheath when the sheath is removed from the housing.

18. The housing of claim 17, wherein the lower cover includes at least one locking pin, and wherein the upper cover includes at least one apertures configured to engage the at least one locking pin.

19. The housing of claim 18, wherein the at least one locking pin and the at least one aperture are configured to engage one another in a press fit engagement.

20. The housing of claim 17, wherein the cover defines an arrow-shaped recess.
